(19) United States
(12) Patent Application Publication
(10) Pub. No.: US 2022/0087561 A1
(43) Pub. Date: Mar. 24, 2022

(54) COMPARTMENT-SPECIFIC DOWN-SCALING OF HIGH-FREQUENCY CONDUCTIVITY TO LOW-FREQUENCY CONDUCTIVITY FOR EEG

(71) Applicant: KONINKLIJKE PHILIPS N.V., EINDHOVEN (NL)

(72) Inventors: ULRICH WOLFGANG KATSCHER, NORDERSTEDT (DE); FABIAN WENZEL, HAMBURG (DE)

(21) Appl. No.: 17/422,568

(22) PCT Filed: Jan. 14, 2020
(86) PCT No.: PCT/EP2020/050742

§ 371 Date: Jul. 13, 2021

Related U.S. Application Data
(60) Provisional application No. 62/792,105, filed on Jan. 14, 2019.

(51) Int. Cl. A61B 5/053; A61B 5/369; A61B 5/00

(52) U.S. Cl. CPC A61B 5/053; A61B 5/7235; A61B 5/369

(57) ABSTRACT

In an embodiment, deriving low frequency conductivity based on radio frequency conductivity derived from Electric Properties Tomography for use in source localization.

### FIELD OF THE INVENTION

The present invention is generally related to equipment for detection of brain disorders, and in particular, conductivity processing of a head region.

### BACKGROUND OF THE INVENTION

Electro-encephalography (EEG) enables the localization of sources responsible for the signals measured at a patient's scalp, which is useful information for facilitating treatment of brain disorders (e.g., surgically removing pathologic sources). The source localization typically requires the knowledge of electric tissue conductivity throughout the head to model propagation of electric signals inside the head.

Electric Properties Tomography (EPT) is a technique to measure electric conductivity a of tissue non-invasively and quantitatively in vivo by numerically post-processing dedicated magnetic resonance (MR) images, using standard MR systems and standard MR sequences.

Conductivity varies with frequency of applied electric currents/electric fields. EEG source localization typically requires knowledge of conductivity at low frequencies (LF) around 1 kHz. EPT yields conductivity at radio frequency (RF) around 100 MHz (Larmor frequency). Accordingly, conductivities obtained from EPT cannot be applied directly for EEG source localization. Therefore, conductivity values are currently taken from the literature. Conductivities obtained from Electric Impedance Tomography (EIT) could be applied directly for EEG source localization, but suffer from low spatial resolution and an ill-posed inverse problem to be solved for EIT.

### SUMMARY OF THE INVENTION

One object of the present invention is to provide patient specific, low frequency conductivity data for source localization. To better address such concerns, in a first aspect of the invention, a computing device configured to derive low frequency conductivity based on measured radio frequency conductivity derived from Electric Properties Tomography (EPT) for use in source localization. Accordingly, patient-specific conductivity data from EPT techniques is enabled for application to source localization.

In one embodiment, the computing device segments areas of a head by segmenting tissue classes in a head for which one or more tissue-specific transfer functions are applied.

In one embodiment, the computing device parameterizes frequency dependence of electric properties by superposing four of the Cole-Cole models, each of the four Cole-Cole models corresponding to a different dispersion frequency.

In one embodiment, the computing device derives the low frequency conductivity from the measured radio frequency conductivity by applying a ratio of conductivities of the four Cole-Cole models from the measured radio frequency conductivity.

In one embodiment, the computing device derives the low frequency conductivity from the measured radio frequency conductivity by reducing a number of the frequency dispersions corresponding to a reduction in the parameters.

In one embodiment, the computing device derives the low frequency conductivity from the measured radio frequency conductivity by acquiring multiple EPT scans.

In one embodiment, the computing device derives the low frequency conductivity from the measured radio frequency conductivity by measuring radio frequency permittivity.

These and other aspects of the invention will be apparent from and elucidated with reference to the embodiment(s) described hereinafter.

### BRIEF DESCRIPTION OF THE DRAWINGS

Many aspects of the invention can be better understood with reference to the following drawings, which are diagrammatic. The components in the drawings are not necessarily to scale, emphasis instead being placed upon clearly illustrating the principles of the present invention. Moreover, in the drawings, like reference numerals designate corresponding parts throughout the several views.

FIG. 1 is a block diagram that illustrates an example conductivity down-scaling system, in accordance with an embodiment of the invention.

FIG. 2 is a block diagram that illustrates an example computing device of FIG. 1, in accordance with an embodiment of the invention.

FIG. 3 is an example diagram that illustrates example conductivity of cortical bone as function of frequency based on implementation of a conductivity down-scaling method, in accordance with an embodiment of the invention.

FIG. 4 is an example diagram that illustrates example permittivity of cortical bone as function of frequency based on implementation of a conductivity down-scaling method, in accordance with an embodiment of the invention.

FIG. 5 is a flow diagram that illustrates an example conductivity down-scaling method, in accordance with an embodiment of the invention.

### DETAILED DESCRIPTION OF EMBODIMENTS

Disclosed herein are certain embodiments of a conductivity down-scaling system and method that estimate low-frequency (LF) conductivity (for Electro-encephalography or EEG) from radio frequency (RF) conductivity (from Electric Properties Tomography or EPT). In one embodiment, a conductivity down-scaling method comprises three steps: (1) measuring RF conductivity with EPT using a standard magnetic resonance (MR) system and with standard MR sequences, (2) segmentation of head (e.g., brain) areas differing in conductivity, and (3) applying a Cole-Cole model for deriving LF conductivity from RF conductivity for each segmented head area separately. Among the various applications for which certain embodiments of a conductivity down-scaling system may be deployed, one particular application is to increase source localization (e.g., of electric foci in the brain) of epilepsy patients, as improved input for surgical removal of these foci. Other applications may involve facilitating surgical procedures for various other brain abnormalities/pathologies.

Digressing briefly, EEG source reconstruction requires knowledge of conductivity at low frequency of different head/brain compartments (grey matter, white matter, ventricles, various nuclei, etc.). EPT yields conductivity of these compartments at radio (Larmor) frequency. Down-scaling of conductivity from radio to low frequency today is typically done by applying compartment-specific parameters from the literature, and hence conductivity values are neither patient-specific nor match individual true conductivity characteristics. In contrast, certain embodiments of a conductivity down-scaling system and method derive personalized, voxel-wise conductivity values via combing EPT measurements with head (e.g., brain) scan segmentation and tissue classification, improving modelling the propagation of electric signals in the head.

Having summarized certain features of a conductivity down-scaling system of the present disclosure, reference will now be made in detail to the description of a conductivity down-scaling system as illustrated in the drawings. While a conductivity down-scaling system will be described in connection with these drawings, there is no intent to limit it to the embodiment or embodiments disclosed herein. Further, although the description identifies or describes specifics of one or more embodiments, such specifics are not necessarily part of every embodiment, nor are all of any various stated advantages necessarily associated with a single embodiment. On the contrary, the intent is to cover all alternatives, modifications and equivalents included within the principles and scope of the disclosure as defined by the appended claims. Further, it should be appreciated in the context of the present disclosure that the claims are not necessarily limited to the particular embodiments set out in the description.

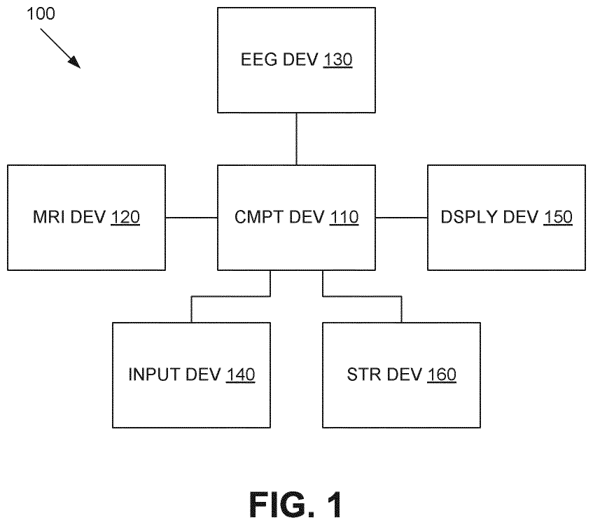

Referring now to FIG. 1, shown is an embodiment of an example conductivity down-scaling system 100. The conductivity down-scaling system 100 includes a computing device 110 connected to and/or in electrical communication with a magnetic resonance imaging (MRI) device 120, an Electro-encephalography (EEG) device 130, a user input device 140, a display device 150, and a storage device 160. The computing device 110 may be a workstation, tablet, laptop, or a controller that serves as an interface between the MRI device 120, the EEG device 130, and the display device 150. The computing device 110 includes one or more processors, memory, and input/output (I/O) interfaces. The memory may be a non-transitory, computer readable storage medium encoded with software that, when executed by the one or more processors, enables the functionality of the conductivity down-scaling system 100 to be implemented. In some embodiments, one or more of the functionality of the conductivity down-scaling system 100 may be implemented in distributed fashion (e.g., via the computing device 110 and one or more remote computing devices). In one embodiment, the computing device 110 controls and accesses data of the EEG device 130, and in some embodiments, the computing device 110 accesses data obtained by the use of the EEG device 130 but does not directly control operations of the EEG device 130. In some embodiments, the computing device 110 receives data from the EEG device 130, processes (e.g., source localization) the same to determine electrical activity levels, and outputs the activity levels to the display device 150 and/or uses in the software to implement functionality of the conductivity down-scaling system 100. In some embodiments, the MRI device 120 operates in different modalities, including but not limited to magnetic resonance imaging, diffusion tensor imaging (DTI), and functional magnetic resonance imaging (fMRI) and outputs imaging data to the computing device 110. In some embodiments, the MRI device 120 operates in different modalities at the same time. The user input device 140 serves as an interface between a user and the computing device 110 and allows the user to interact with the computing device 110 by enabling the entering of user inputs. The user input device 140 can be a keyboard, a camera, a scanner, a mouse, a touchpad, a trackpad, a touchscreen mounted on the display device 150, a communication port, a USB port, a hand gesture control device, or a virtual reality glove.

The computing device 110 performs several functions. In some embodiments, the computing device 110 receives magnetic resonance data from the MRI device 120, processes the same, and outputs the magnetic resonance image data to the display device 150 for display of the magnetic resonance images. In some embodiments, the computing device 110 comprises software that enables Electric Properties Tomography (EPT). Digressing briefly, EPT derives a patient's electric properties (e.g., EPs, including conductivity and permittivity) using the magnetic resonance data and standard magnetic resonance sequences. EPT does not apply externally mounted electrodes, currents, or radio frequency (RF) probes. In EPT, eddy currents induced by application of magnetic RF fields (e.g., at the Larmor frequency, typically around 100 MHz) via standard magnetic resonance systems and magnetic resonance sequences are used in the imaging technique of EPT. More particularly, the conductivity and permittivity of the patient subject to the MRI device 120 distorts B1 (a component of the magnetic RF field responsible for spin excitation). Measurements of this distorted B1 field distribution enables the reconstruction of the EPs causing the observed distortions, enabling access to the desired electrical tissue properties. In effect, EPT is quantitative magnetic resonance, yielding absolute values of conductivity and permittivity. As indicated above, EPT is based on eddy currents induced by magnetic RF fields. The resulting EPs, which generally depend on the applied frequency, belong to the MHz frequency range corresponding to the Larmor frequency of the current main field strength. The physical equations underlying EPT and the basic reconstruction techniques resulting directly from these physical equations may be found in Katscher U, van den Berg CAT. Electric properties tomography: Biochemical, physical and technical background, evaluation and clinical applications. NMR Biomed. 2017; 30: 3729, incorporated herein by reference in its entirety.

In some embodiments, the magnetic resonance data may be T1 weighted (T1 W) magnetic resonance images and the computing device 110 may automatically segment the magnetic resonance image (based on a segmentation protocol) to delineate geometries of anatomical structures in or of the head (e.g., brain) of a patient. In some instances, the computing device 110 may automatically segment the anatomic structures from the magnetic resonance data based on a three-dimensional (3D) model of a head/brain or a patient body. In instances where the patient body includes a brain, the computing device 110 may receive a 3D brain model from storage through a wired or wireless connection to a server or a remote workstation where the 3D brain model is stored. In some implementations, the 3D brain model may be stored in a database of the storage device 160 or a storage device retrievable by the computing device 110. In some instances, the 3D brain model is a shape-constrained deformable brain model. For instance, and as described in Wenzel F et al., Rapid fully automatic segmentation of subcortical brain structures by shape-constrained surface adaptation. Medical Image Analysis 46 (2018) 146, incorporated herein by reference in its entirety, brain structures (e.g., subcortical) in T1-weighted magnetic resonance imaging data may be segmented using a shape-constrained deformable surface model. Digressing briefly, shape-constrained deformable models combine properties of deformable models or active contour models and active shape models. In shape-constrained deformable models, organ surfaces are represented by a triangulated mesh of fixed topology, where a shape model is created using principle component analysis (PCA) from a set of training shapes or via a piece-wise linear transformation model (e.g., for complex anatomies). Generally, as explained in the Wenzel F et al document, the method involves initialization, parametric adaptation, deformable adaptation, boundary detection, and boundary detection training. In some instances, the 3D brain model may be the brain model described in “Evaluation of traumatic brain injury patients using a shape-constrained deformable model,” by L. Zagorchev, C. Meyer, T. Stehle, R. Kneser, S. Young and J. Weese, 2011, in Multimodal Brain Image Analysis by Liu T., Shen D., Ibanez L., Tao X. (eds). MIA 2011. Lecture Notes in Computer Science, vol 7012. Springer, Berlin, Heidelberg, the entirety of which is hereby incorporated by reference. In some instances, the 3D brain model may be the deformable brain model described in U.S. Pat. No. 9,256,951, titled “SYSTEM FOR RAPID AND ACCURATE QUANTITATIVE ASSESSMENT OF TRAUMATIC BRAIN INJURY” or the shape-constrained deformable brain model described in U.S. Pat. App. Pub. No. 20150146951, titled “METHOD AND SYSTEM FOR QUANTITA FIVE EVALUATION OF IMAGE SEGMENTATION,” each of which is hereby incorporated by reference in its entirety.

In some embodiments, the storage device 160 stores historical and statistical data about imaged head (e.g., brain) regions and incidental findings and/or anomalies. In some implementations, the storage device 160 may comprise patient information, including gender, race, and age of the patient, patient historical data, etc. It is noted that while the storage device 160 is depicted as a device local to the computing device 110, the functionality of the storage device 160 may be realized via a remote database or server connected to the computing device 110 by wire or coupled wirelessly. In some embodiments, the storage device 160 can be part of a cloud-based service provided by a third-party medical database service provider.

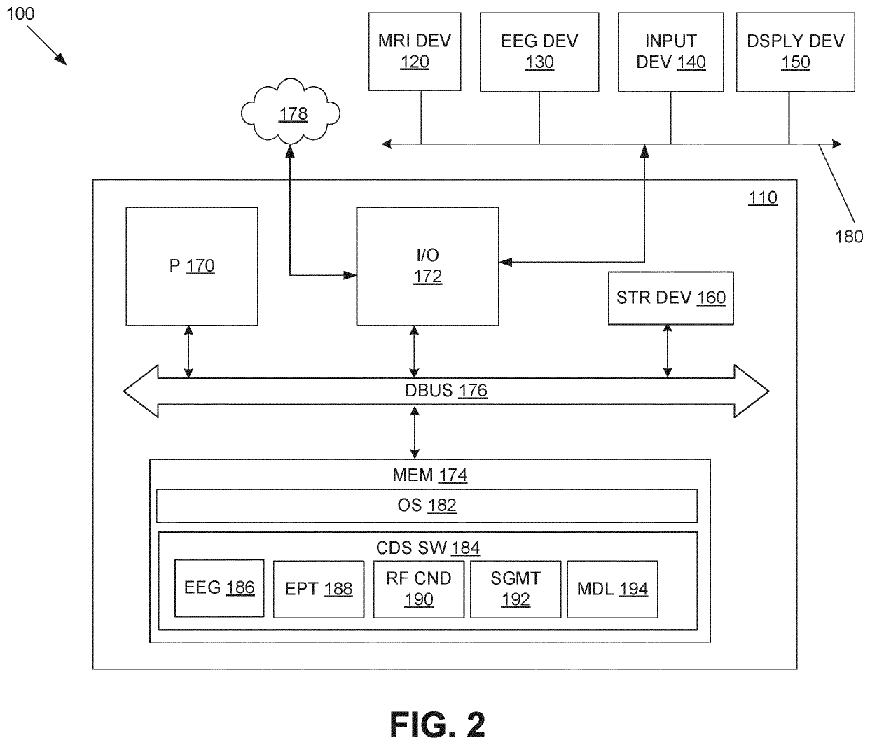

Having described example components of a conductivity down-scaling system 100, attention is directed to FIG. 2, which illustrates an example architecture for the computing device 110 as used in the conductivity down-scaling system 100. One having ordinary skill in the art should appreciate in the context of the present disclosure that the example computing device 110 is merely illustrative of one embodiment, and that some embodiments of computing devices may comprise fewer or additional components, and/or some of the functionality associated with the various components depicted in FIG. 2 may be combined, or further distributed among additional modules or computing devices located locally or both locally and remotely, in some embodiments. The computing device 110 is depicted in this example as a computer system, such as one providing a function of an application server. It should be appreciated that certain well-known components of computer systems are omitted here to avoid obfuscating relevant features of the computing device 110. In one embodiment, the computing device 110 comprises one or more processors, with one shown as processor 170, input/output (I/O) interface(s) 172, and memory 174, all coupled to one or more data busses, such as data bus 176. The memory 174 may include any one or a combination of volatile memory elements (e.g., random-access memory RANI, such as DRAM, and SRAM, etc.) and nonvolatile memory elements (e.g., ROM, Flash, solid state, EPROM, EEPROM, hard drive, tape, CDROM, etc.). The memory 174 may store a native operating system, one or more native applications, emulation systems, or emulated applications for any of a variety of operating systems and/or emulated hardware platforms, emulated operating systems, etc. In one embodiment, the storage device 160 is coupled directly to the data bus 176, though in some embodiments, may be connected to the computing device 110 via the I/O interfaces 172, or connected via another network (e.g., the Internet) 178, such as a network connected device. In some embodiments, the network 178 may comprise one or more wired and/or wireless networks, including a telephone exchange network, wireless (e.g., Wireless Fidelity or Wi-Fi) network, a local network (Local Area Network or LAN, personal area network or PAN), the Internet, a metropolitan area network (MAN), and/or a cellular network, among others. Also connected to the computing device 110 via the I/O interfaces 172 are the MRI device 120, the EEG device 130, user input device 140, and display device 150. In some embodiments, these devices are coupled via a network 180, which may comprise a wired and/or wireless network (e.g., Bluetooth, LAN, etc.). It should be appreciated by one having ordinary skill in the art, in the context of the present disclosure, that variations in the manner of connections of these devices to the computing device 110 may be implemented. For instance, the user input device 140 may be connected directly to the data bus 176.

The storage device 160 may be embodied as persistent memory (e.g., optical, magnetic, and/or semiconductor memory and associated drives). In some embodiments, as noted above, the storage device 160 may be replaced with a network-connected storage device, including one maintaining a third party database, such as a medical facility database.

In the embodiment depicted in FIG. 2, the memory 174 comprises an operating system (OS) 182, and conductivity down-scaling software 184, which includes EEG module 186, EPT module 188, RF conductivity module 190, segmentation module 192, and a Cole-Cole Model module 194, which collectively provide, in one embodiment, functionality of the conductivity down-scaling system 100. In one embodiment, the EEG module 186 processes the EEG data from the EEG device 130. In some embodiments, functionality of the EEG module 186 may reside in the EEG device 130, and the results of computation at the EEG device 130 are communicated to the conductivity down-scaling software 184. The EPT module 188 provides EPT processing of the magnetic resonance data and magnetic resonance sequences. A description of the balance of the modules 190-194 are described below under corresponding headings. In some embodiments, functionality may be distributed among the computing device 110 and one or more devices of the conductivity down-scaling system 100. In some embodiments, functionality of the conductivity down-scaling system 100 may be distributed among plural devices in local and remote locations. For instance, one or more of the functionality of the conductivity down-scaling system 100 may be implemented at least in part using a cloud computing platform. Note that memory 174 may include one or more additional functionality, including one or more APIs, communications software, etc.

Execution of the conductivity down-scaling software 184 (including associated modules, which include executable code, or generally, instructions) may be implemented by the processor 170 (or processors) under the management and/or control of the operating system 182. Further description of the conductivity down-scaling software 184 is described below.

The processor 170 may be embodied as a custom-made or commercially available processor, a central processing unit (CPU) or an auxiliary processor among several processors, a semiconductor based microprocessor (in the form of a microchip), a macroprocessor, one or more application specific integrated circuits (ASICs), a plurality of suitably configured digital logic gates, and/or other well-known electrical configurations comprising discrete elements both individually and in various combinations to coordinate the overall operation of the computing device 66.

The I/O interfaces 172 comprise hardware and/or software to provide one or more interfaces to devices coupled to one or more networks 178, 180. In other words, the I/O interfaces 172 may comprise any number of interfaces for the input and output of signals (e.g., analog or digital data) for conveyance of information (e.g., data) over various networks and according to various protocols and/or standards.

When certain embodiments of the computing device 110 are implemented at least in part with software (including firmware, op-code, middleware, etc.), as depicted in FIG. 2, it should be noted that the software (e.g., such as the conductivity down-scaling software 184 and associated modules 186, 188, 190, 192, and 194) can be stored on a variety of non-transitory computer-readable (storage) medium for use by, or in connection with, a variety of computer-related systems or methods. In the context of this document, a computer-readable medium may comprise an electronic, magnetic, optical, or other physical device or apparatus that may contain or store a computer program (e.g., executable code or instructions) for use by or in connection with a computer-related system or method. The software may be embedded in a variety of computer-readable mediums for use by, or in connection with, an instruction execution system, apparatus, or device, such as a computer-based system, processor-containing system, or other system that can fetch the instructions from the instruction execution system, apparatus, or device and execute the instructions. Note that the various modules described herein may comprise executable code or instructions, though in some embodiments, functionality of one or more modules may be implemented all, or in part, using hardware.

When certain embodiments of the computing device 110 are implemented at least in part with hardware, such functionality may be implemented with any or a combination of the following technologies, which are all well-known in the art: a discrete logic circuit(s) having logic gates for implementing logic functions upon data signals, an application specific integrated circuit (ASIC) having appropriate combinational logic gates, a programmable gate array(s) (PGA), a field programmable gate array (FPGA), relays, contactors, etc.

Having described the underlying system components for certain embodiments of a conductivity down-scaling system 100, attention is directed below to a description of functionality implemented by the conductivity down-scaling software 184 in conjunction with one or more of the devices of the conductivity down-scaling system 100.

Measuring RF Conductivity with EPT

RF conductivity σ_{RF} is determined by applying the simplified Helmholtz equation

σ_{RF}=Δφ,/(2ωμ₀) Eq. (1)

to the transceive phase φ obtained via (preferably off-resonance independent) magnetic resonance scans like spin-echo based sequences, Steady-State-Free-Precession (SSFP) sequences, or (particularly for bone/bone marrow) ultrashort/zero echo time (UTE/ZTE) sequences. In Eq. (1), the Larmor frequency is given by ω, μ₀ is the magnetic permeability of free space, and Δ is the Laplace operator. The measurement can be repeated at different main field strengths B0 (typically 0.5 T/1.5 T/3 T/7 T) related to different ω (21 MHz/64 MHz/128 MHz/300 MHz).

Segmentation of Head Areas

Segmentation of tissue classes in the head, for which tissue-specific transfer functions for downscaling EPT results may be applied, may be performed according to one of many ways. In one embodiment, multi-atlas segmentation techniques are applied, in which the most promising tissue class is selected according to a voting scheme related to corresponding voxels of a number of different tissue atlases, which have been registered to the magnetic resonance imaging scan of interest. In some embodiments, shape-constrained, model-based adaptation of tissue boundary surfaces can be applied, as described further in the Wenzel F et al document referenced above. In some embodiments, the described approaches may be implemented according to (a combination of) deep learning techniques.

Applying the Cole-Cole Model

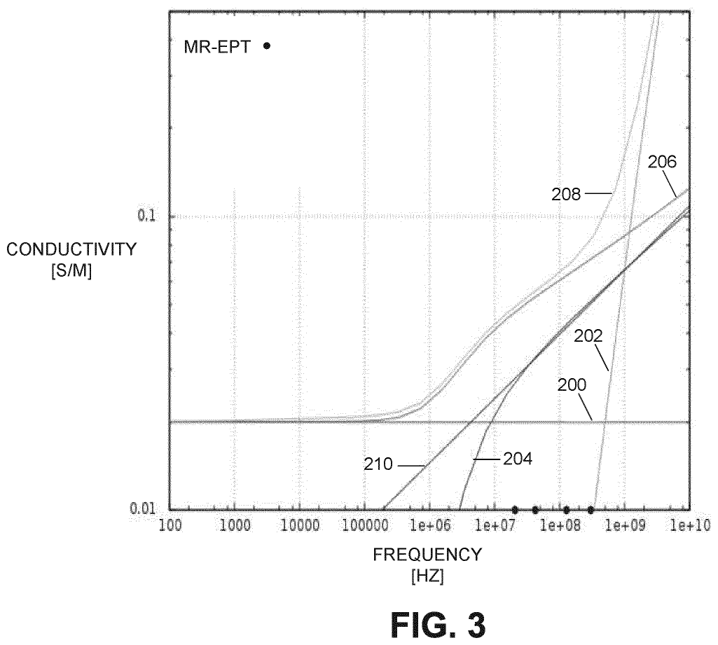

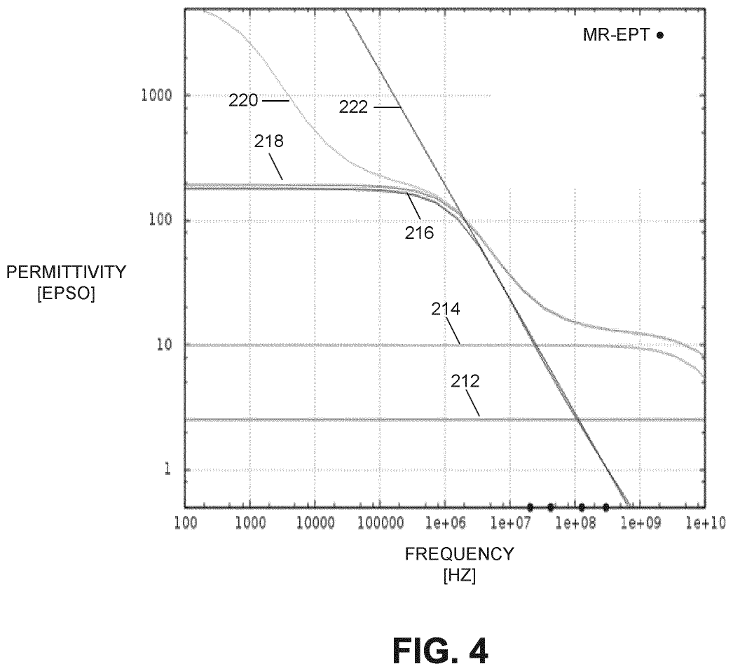

Frequency dependence of electric properties has been parametrized by superposing four Cole-Cole models, each describing a dispersion at a different frequency (see FIGS. 3-4 for the example of cortical bone).

$${ɛ}_{r} ( ω ) + \frac{i σ ( ω )}{{ωɛ}_{0}} = {ɛ}_{∞} + {Σ}_{n \leq 4} \frac{{Δɛ}_{n}}{1 + {( i {ωτ}_{n} )}^{( 1 - {α}_{n} )}} + \frac{i {σ}_{0}}{{ωɛ}_{0}} , \qquad Eq . 2$$

with ε₀ the permittivity of free space. The remaining 14 parameters (ε_{∞}, Δεₙ, τₙ, αₙ, σ₀) depend on the specific tissue type and have been investigated in, for instance, Gabriel S et al., The dielectric properties of biological tissues: III. Parametric models for the dielectric spectrum of tissues. Phys Med Biol. 1996; 41: 2271, which is incorporated herein by reference in its entirety. Furthermore, it is expected that these 14 parameters vary from subject to subject, and thus, should be determined for each patient individually. The frequencies related to the four described dispersions are given by 1/τₙ as shown in Table 1 below:

TABLE 1

| | | |
|---|---|---|
| n = 1 | α-dispersion | around 100 Hz, ionic diffusion |
| | | processes on both sides of cell membrane |
| n = 2 | β-dispersion | around 10 kHz, polarisation of cell |
| | | membrane/cellular macro-molecules |
| n = 3 | γ-dispersion | around 10 MHz, polarisation of water molecules |
| n = 4 | δ-dispersion | around 100 GHz, other minor processes |

Conductivity and permittivity of Eq. (2) can be written separately as follows:

$$\begin{matrix}σ ( ω ) = {σ}_{0} + {Σ}_{n \leq 4} \frac{{Δɛ}_{n} {s}_{n}}{{s}_{n}^{2} + {p}_{n}^{2}} {ωɛ}_{0} & Eq . 3 a \\ {ɛ}_{r} ( ω ) = {ɛ}_{∞} + {Σ}_{n \leq 4} \frac{{Δɛ}_{n} {p}_{n}}{{s}_{n}^{2} + {p}_{n}^{2}} \\ with \\ {s}_{n} ( ω ) = Im ( {i}^{1 - {α}_{n}} ) {( {ωτ}_{n} )}^{1 - {α}_{n}} \\ {p}_{n} ( ω ) = Re ( {i}^{1 - {α}_{n}} ) {( {ωτ}_{n} )}^{1 - {α}_{n}} + 1 , & Eq . 3 b\end{matrix}$$

each with 13 parameters left to be determined per tissue type and patient.

In one embodiment, the conductivity down-scaling software 184 may implement four different ways to derive σ_{LF} from σ_{RF} via Eq. (3a) as listed in the following, ordered from the simplest approach (A) to most complex approach (D). Since Eq. (3a), with its 13 free parameters, cannot be solved exactly, one goal is to simplify Eq. (3a) and determine the reduced number of remaining free parameters.

Approach (A)—Straight-Forward Conductivity Ratio

Apply ratio from conductivities of the 4-Cole-Cole model (σ^{CC}) to conductivity from EPT (σ^{EPT}):

$${σ}_{LF} = \frac{{σ}_{LF}^{CC}}{{σ}_{RF}^{CC}} {σ}^{EPT} \qquad Eq . 4$$

Approach (B)—Reduce Number of Dispersions

It is analyzed via Eq. (3), which dispersions are relevant for the frequency range LF to RF. FIG. 3 is an example diagram that illustrates conductivity of cortical bone as function of frequency based on one implementation of an embodiment of conductivity down-scaling method, and in particular, according to the 4-Cole-Cole model set forth in and taken from the Gabriel S et al. document referenced above. The following reference numbers denote that corresponding lines: 202 is sigma_0, 202 is delta-dispersion, 204 is gamma-dispersion, 206 is sigma_0+gamma, 208 is the total conductivity including all terms of Eq. (2), and 210 is a Polynomial fit ˜ω^{c} with c=α₄ for gamma-dispersion at Larmor frequencies. The lines 200, 202, and 204 correspond to selected terms of Eq. (2). Line 206 corresponds to a superposition of selected terms determining conductivity between 1 kHz (EEG frequency) and Larmor frequencies of today's standard magnetic resonance systems (indicated by black dots, ⋅). As shown in FIG. 3, only gamma-dispersion and σ₀ are required to sufficiently approximate this frequency range for cortical bone, thus skipping 9 out of 13 free parameters. Via measuring σ_{RF}^{EPT}, one of the remaining 4 free parameters may be determined, and the other 3 are taken from literature (see, e.g., the Gabriel S et al. document referenced above). As visible from FIG. 3, for cortical bone, the contribution of the gamma-dispersion has to be removed from σ_{RF}^{EPT} yielding σ₀, which is equivalent with au required for EEG.

Approach (C)—Multiple EPT Scans

Following the approach of (B), σ_{RF}^{EPT} may be measured for different frequencies using multiple magnetic resonance systems with different main field strength B0 (or a single MR system with rampable field strength, such as disclosed in Dixon A K et al., MR imaging using a rampable system. J Comp Assist Tom 1988; 12: 903, incorporated herein by reference in its entirety). The range of today's standard MR systems is indicated in FIG. 3. Ideally, using four different B0, all 4 free parameters may be determined. In practice, gamma-dispersion for the measured frequency range may be approximated by a simple polynomial σ₄(ω)˜ω^{c} (see FIG. 3) with c=α₄, which makes the fitting procedure more stable. Acquiring multiple EPT scans, in one embodiment, image segmentation and EPT reconstruction are performed for each scan separately, and subsequently to register the obtained σ_{RF}^{EPT} for the different B0.

Approach (D)—Include Permittivity

Following the approaches of (B, C), not only may σ_{RF}^{EPT} be measured at one or more different frequencies, but also ε_{RF}^{EPT} (see, e.g., the Katscher, van den Berg document referenced above). FIG. 4 is an example diagram that illustrates example permittivity of cortical bone as function of frequency based on implementation of an embodiment of conductivity down-scaling method, and in particular, according to the 4-Cole-Cole model taken from the Gabriel S et al. document referenced above. The following reference numbers denote the corresponding lines: 212 is eps_Inf, 214 is delta-dispersion, 216 is gamma-dispersion, 218 is eps_inf+delta+gamma, 220 is the total permittivity including all terms of Eq. (2), and 222 is a polynomial fit ω^{1-c} again with c=α₄ for gamma-dispersion at Larmor frequencies. Note that lines 212, 214, and 216 correspond to selected terms of Eq. (2). Line 218 corresponds to a superposition of selected terms determining conductivity between 1 kHz (EEG frequency) and Larmor frequencies of today's standard magnetic resonance systems (indicated by black dots, ⋅). In the frequency range of today's standard magnetic resonance systems (as indicated in FIG. 3), gamma-dispersion and delta-dispersion (constant for magnetic resonance frequencies) are relevant for cortical bone's permittivity. As for conductivity, gamma-dispersion for the measured frequency range can be approximated by a simple polynomial ε₄(ω)˜ω^{1-c} (see FIG. 3). Thus, including RF permittivity measurements further stabilizes the fitting procedure of conductivity.

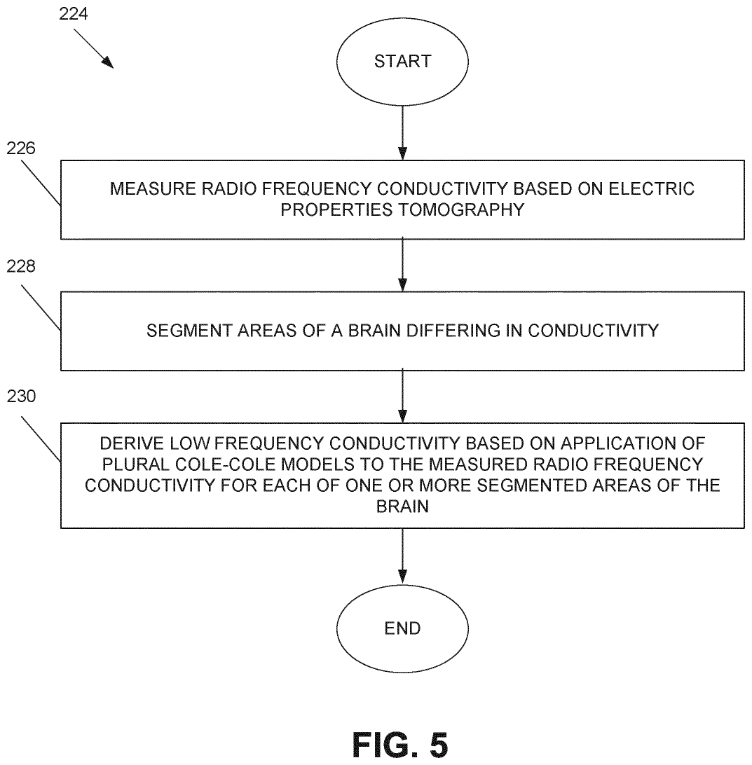

Having described certain functionality of the conductivity down-scaling system 100 (e.g., conductivity down-scaling software 184) illustrated in FIG. 2, it should be appreciated that one embodiment of an example conductivity down-scaling method, depicted in FIG. 5 and denoted as method 224, which is shown bounded by start and end, comprises measuring radio frequency conductivity based on Electric Properties Tomography (226); segmenting areas of a head (e.g., brain) differing in conductivity (228); and deriving low frequency conductivity based on application of plural Cole-Cole models to the measured radio frequency conductivity for each of one or more segmented areas of the head (230).

Any process descriptions or blocks in flow diagrams should be understood as representing modules, segments, or portions of code which include one or more executable instructions for implementing specific logical functions or steps in the process, and alternate implementations are included within the scope of the embodiments in which steps/functions may be omitted, added, and/or executed out of order from that shown or discussed, including substantially concurrently or in reverse order, depending on the functionality involved, as would be understood by those reasonably skilled in the art of the present disclosure.

In one embodiment, a computing device is disclosed, comprising: a memory comprising instructions; and one or more processors configured to execute the instructions to: measure radio frequency conductivity based on Electric Properties Tomography; segment areas of a head differing in conductivity; and derive low frequency conductivity based on application of plural Cole-Cole models to the measured radio frequency conductivity for each of one or more segmented areas of the head, the low frequency conductivity applicable to source localization.

In one embodiment, the preceding computing device, wherein the one or more processors are further configured to execute the instructions to receive magnetic resonance data and apply the Electric Properties Tomography to the magnetic resonance data.

In one embodiment, any one of the preceding computing devices, wherein the one or more processors are further configured to execute the instructions to apply the derived low frequency conductivity to electro-encephalography data for the source localization.

In one embodiment, any one of the preceding computing devices, wherein the derived low frequency conductivity applied to the electro-encephalography data is patient-specific.

In one embodiment, any one of the preceding computing devices, wherein the low frequency conductivity comprises a frequency at or proximal to 1000 kilohertz and the measured radio frequency conductivity comprises a frequency at or proximal to 100 megahertz.

In one embodiment, any one of the preceding computing devices, wherein the one or more processors are further configured to execute the instructions to segment areas of a head by segmenting tissue classes in a head for which one or more tissue-specific transfer functions are applied.

In one embodiment, any one of the preceding computing devices, wherein the one or more processors are further configured to execute the instructions to: parameterize frequency dependence of electric properties by superposing four of the Cole-Cole models, each of the four Cole-Cole models corresponding to a different dispersion frequency.

In one embodiment, any one of the preceding computing devices, wherein the one or more processors are further configured to execute the instructions to derive the low frequency conductivity from the measured radio frequency conductivity by applying a ratio of conductivities of the four Cole-Cole models from the measured radio frequency conductivity.

In one embodiment, any one of the preceding computing devices, wherein the one or more processors are further configured to execute the instructions to derive the low frequency conductivity from the measured radio frequency conductivity by reducing a number of the frequency dispersions corresponding to a reduction in the parameters.

In one embodiment, any one of the preceding computing devices, wherein the one or more processors are further configured to execute the instructions to derive the low frequency conductivity from the measured radio frequency conductivity by acquiring multiple EPT scans.

In one embodiment, any one of the preceding computing devices, wherein the one or more processors are further configured to execute the instructions to derive the low frequency conductivity from the measured radio frequency conductivity by measuring radio frequency permittivity.

In one embodiment, a computer-implemented method is disclosed that implements functionality of any one of the preceding computing devices.

In one embodiment, a non-transitory, computer readable storage medium is disclosed that comprises instructions encoded thereon, wherein when the instructions are executed by one or more processors, causes the one or more processors to implement a method corresponding to functionality of any one of the preceding computing devices.

In one embodiment, a system is disclosed that implements functionality of any one of the preceding computing devices.

In one embodiment, a computer-implemented method is disclosed, comprising: measuring radio frequency conductivity based on Electric Properties Tomography; segmenting areas of a head differing in conductivity; and deriving low frequency conductivity based on application of plural Cole-Cole models to the radio frequency conductivity for each of one or more segmented areas of the head, the low frequency conductivity applicable to source localization.

In one embodiment, the preceding method, further comprising applying the derived low frequency conductivity to electro-encephalography data for source localization.

In one embodiment, a non-transitory, computer readable storage medium is disclosed comprising instructions encoded thereon, wherein when the instructions are executed by one or more processors, causes the one or more processors to: measure radio frequency conductivity based on Electric Properties Tomography; segment areas of a head differing in conductivity; and derive low frequency conductivity based on application of plural Cole-Cole models to the measured radio frequency conductivity for each of one or more segmented areas of the head, the low frequency conductivity applicable to source localization.

In one embodiment, the prior non-transitory, computer readable storage medium claim, wherein when the instructions are executed by one or more processors, causes the one or more processors to: apply the derived low frequency conductivity to electro-encephalography data for source localization.

In one embodiment, a system is disclosed, comprising: a magnetic resonance imaging device; and a computing device comprising a memory comprising instructions and one or more processors configured to execute the instructions to: measure radio frequency conductivity based on Electric Properties Tomography; segment areas of a head differing in conductivity; and derive low frequency conductivity based on application of plural Cole-Cole models to the measured radio frequency conductivity for each of one or more segmented areas of the head, the low frequency conductivity applicable to source localization.

In one embodiment, the preceding system, further comprising an electro-encephalography device, wherein the one or more processors are further configured to execute the instructions to: apply the derived low frequency conductivity to electro-encephalography data for source localization.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

Note that various combinations of the disclosed embodiments may be used, and hence reference to an embodiment or one embodiment is not meant to exclude features from that embodiment from use with features from other embodiments. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical medium or solid-state medium supplied together with or as part of other hardware, but may also be distributed in other forms. Note that memory used to store instructions (e.g., application software) in one or more of the devices of the environment may be referred to also as a non-transitory computer-readable medium. Any reference signs in the claims should be not construed as limiting the scope.

1. A computing device comprising:
a memory configured to store instructions; and
one or more processors configured to execute the instructions to:
measure radio frequency conductivity based on Electric Properties Tomography;
segment areas of a head differing in conductivity; and
derive low frequency conductivity based on application of plural Cole-Cole models to the measured radio frequency conductivity for each of one or more segmented areas of the head, the low frequency conductivity applicable to source localization.

2. The computing device claim 1, wherein the one or more processors are further configured to execute the instructions to receive magnetic resonance data and apply the Electric Properties Tomography to the magnetic resonance data.

3. The computing device of claim 1, wherein the one or more processors are further configured to execute the instructions to apply the derived low frequency conductivity to electro-encephalography data for the source localization.

4. The computing device claim 1, wherein the derived low frequency conductivity applied to the electro-encephalography data is patient-specific.

5. The computing device of claim 1, wherein the low frequency conductivity comprises a frequency at or proximal to 1000 kilohertz and the measured radio frequency conductivity comprises a frequency at or proximal to 100 megahertz.

6. The computing device of claim 1, wherein the one or more processors are further configured to execute the instructions to segment areas of a head by segmenting tissue classes in a head for which one or more tissue-specific transfer functions are applied.

7. The computing device of claim 1, wherein the one or more processors are further configured to execute the instructions to:
parameterize frequency dependence of electric properties by superposing four of the Cole-Cole models, each of the four Cole-Cole models corresponding to a different dispersion frequency.

8. The computing device of claim 1, wherein the one or more processors are further configured to execute the instructions to derive the low frequency conductivity from the measured radio frequency conductivity by applying a ratio of conductivities of the four Cole-Cole models from the measured radio frequency conductivity.

9. The computing device of claim 1, wherein the one or more processors are further configured to execute the instructions to derive the low frequency conductivity from the measured radio frequency conductivity by reducing a number of the frequency dispersions corresponding to a reduction in the parameters.

10. The computing device of claim 1, wherein the one or more processors are further configured to execute the instructions to derive the low frequency conductivity from the measured radio frequency conductivity by acquiring multiple EPT scans.

11. The computing device of claim 1, wherein the one or more processors are further configured to execute the instructions to derive the low frequency conductivity from the measured radio frequency conductivity by measuring radio frequency permittivity.
